(19) United States
(12) Patent Application Publication
(10) Pub. No.: US 2018/0235517 A1
(43) Pub. Date: Aug. 23, 2018

(54) System and Method For Identifying Posture Details and Evaluating Athletes' Performance

(71) Applicant: Anshuman Kumar SINGH, Bangalore (IN)

(72) Inventor: Anshuman Kumar SINGH, Bangalore (IN)

(21) Appl. No.: 15/751,172

(22) PCT Filed: Aug. 10, 2016
(86) PCT No.: PCT/IN2016/000205

§ 371 Date: Feb. 8, 2018

(30) Foreign Application Priority Data
Aug. 10, 2015 (IN) 4148/CHE/2015

(51) Int. Cl. A61B 5/11

(52) U.S. Cl. CPC A61B 5/1116; A61B 5/1118; G06N 7/005; A61B 2503/10; A61B 5/1128

(57) ABSTRACT

The embodiments herein provide a system and method for collecting specific form and posture related visual data from an athlete for analysis. The form-related diagnosis is extracted from an athlete's video recording to establish a correlation between predicted analysis and actual observation. The data related to specific form and posture are collected from a person's pictures taken at specific angles and videos of a person performing a predefined activity for the review by experts. The picture or video of user while performing an exercise/a sport activity is captured. The posture, symmetry and body structure details are identified through visual recognition of bones, muscles or preset points. A quantitative identification/estimation of load-bearing and stress-bearing capability of a body part is done. A personalized grade sensitivity matrix is calculated/computed to provide an intuitive representation of data and multiple outputs with different renderings of data.

### CROSS-REFERENCE TO RELATED APPLICATIONS

The embodiments herein claims the priority of the Indian Provisional Patent Application filed on Aug. 10, 2015 with the number 4148/CHE/2015 and entitled, “SYSTEM AND METHOD FOR IDENTIFICATION OF ATHLETES' PERFORMANCE BASED ON VISUAL INPUTS”, and the contents of which are included in entirety as reference herein.

### BACKGROUND

### Technical Field

The embodiments herein are generally related to a field of sports and exercises. The embodiments herein are particularly related to a system and method for identifying posture details for evaluating a stress or load bearing capability and performance of an athlete based on visual inputs. The embodiments herein are more particularly related to a system and method for collecting specific form and posture related details based on image data or visual data from an athlete for analysis and evaluation of a stress or load bearing capability and performance of an athlete. The embodiments herein are also related to a system and method for collecting specific form and posture related visual data from an athlete for analysis.

### Description of the Related Art

Measuring and analyzing an athlete's performance is critical for identifying and monitoring the athlete's form and fitness. Timely diagnosis of a possible injury and analysis helps athletes in improving their performance.

At present, an athlete performs the activities, which need to be monitored, in a laboratory setting with multiple sensors and equipments connected to the user for accurate measurements and analyses. This requires a physical presence of the athlete at the location of analysis. The currently available methods lack remote means for establishing a correlation between a predicted analysis and an actual observation of an athlete's performance. There are no methods available for remotely identifying qualitative description of a performance of athletes, analyses and rendering of the analyzed data.

Hence, there is a need for a system and method for extracting form-related diagnosis and any existing preconditions from an athlete's visual recording and establishing correlation between predicted analysis and actual observation. There is also a need for a method for remote collection of data for the purpose of review by experts. Further there is a system and method for collecting specific form and posture related details based on image data or visual data from an athlete for analysis and evaluation of a stress or load bearing capability and performance of an athlete

The abovementioned shortcomings, disadvantages and problems are addressed herein and which will be understood by reading and studying the following specification.

### Object of the Embodiments Herein

The primary object of the embodiments herein is to provide a system and method for evaluation of athletes' performance based on visual inputs or images.

Another object of the embodiments herein is to provide a system and method for extracting form related diagnosis from an athlete's video recording and establishing correlation between predicted analysis and actual observation.

Yet another object of the embodiments herein is to provide a system and method for collecting specific form and posture related visual data from an athlete for analysis.

Yet another object of the embodiments herein is to provide a method for remote collection of visual data of an athlete's performance for the purpose of review by experts.

Yet another object of the embodiments herein is to provide a method for simple analysis of an athlete's performance with fewer/lesser variables than conventional data analysis methods.

Yet another object of the embodiments herein is to provide a system and method for collecting specific form and posture related data from a person's picture taken at specific angles.

Yet another object of the embodiments herein is to provide a system and method to identify potential injury points for an athlete by analyzing visual information of the athlete.

Yet another object of the embodiments herein is to provide a system and method to identify potential musculoskeletal strengths and weaknesses of an athlete by analyzing visual information of the athlete.

Yet another object of the embodiments herein is to provide a system and method to identify probable asymmetries in an athlete's body structure and the way they may affect the athlete's performance or predisposition towards injuries.

Yet another object of the embodiments herein is to provide a system and method to establish a framework for making biomechanical analysis framework adaptable based on athlete's body structure, symmetry and posture.

Yet another object of the embodiments herein is to provide a system and method for extracting form-related diagnosis from an athlete's visual recording.

Yet another object of the embodiments herein is to provide a system and method for establishing correlation between predicted analysis and actual observation of an athlete's performance through visual information of the athlete.

Yet another object of the embodiments herein is to provide a system and method for predicting potential injury points in an athlete by analysis of visual information and health parameters of the athlete.

Yet another object of the embodiments herein is to provide a system and method for collecting specific form and posture related details based on image data or visual data from an athlete for analysis and evaluation of a stress or load bearing capability and performance of an athlete during an exercise/sport activity.

These and other objects and advantages of the embodiments herein will become readily apparent from the following summary and the detailed description taken in conjunction with the accompanying drawings.

### SUMMARY

The following details present a simplified summary of the embodiments herein to provide a basic understanding of the several aspects of the embodiments herein. This summary is not an extensive overview of the embodiments herein. It is not intended to identify key/critical elements of the embodiments herein or to delineate the scope of the embodiments herein. Its sole purpose is to present the concepts of the embodiments herein in a simplified form as a prelude to the more detailed description that is presented later.

The other objects and advantages of the embodiments herein will become readily apparent from the following description taken in conjunction with the accompanying drawings.

The various embodiments herein provide a system and method for collecting specific form and posture related visual data from an athlete for analysis. The present embodiments are also related to a system and method for extracting form related diagnosis from an athlete's video recording and establishing correlation between predicted analysis and actual observation.

According to an embodiment herein, a system is provided for collecting, storing and analyzing specific form and posture related visual data from an athlete for predicting injury prone body parts and/or actions through data and visual analysis and simulations. The system comprises an input module configured for capturing pictures or image or video of a user involved in an exercise or sport activity while performing the exercise and sport activity, and wherein the input module is an image capturing device, and wherein the image capturing device is a digital camera or a video camera. A computing system is configured for collecting specific form and posture related data from the captured picture or images or video of the user. A sensor module is provided in the computing system and configured for visually capturing the poses and motion of a user performing an activity or action, and wherein the sensor module comprises a plurality of sensors, and wherein the sensor module is configured to detect and measure a time and magnitude of pressure exerted on the user during the exercise or sport activity and a magnitude of pressure released by the user during a plurality of actions performed by the user. An adaptive assessment module is provided in the computing system to create an adaptive model, and wherein the adaptive assessment module is run on a hardware processor provided in the computing system and configured to create an adaptive model based on the collected specific form and posture related data from the captured picture or images or video of the user visual data and pre-medical conditions provided by the user, and wherein the adaptive assessment module comprises a risk multiplier module, an activity specific stress modeling module and an injury risk measurement module. A handheld computing device is configured for analysing an output data from the adaptive assessment module and rendering results and reports of analysis for predicting injury prone body parts and/or actions, and wherein the hand held computing device is connected to the computing system through a wired or wireless network. An analytics module is provided in the handheld computing device and configured for analysing the data from the sensor module and the adaptive assessment module, and wherein the analytics module is configured to compute a plurality of results that are rendered to the user. A visualization module is provided in the handheld computing device and configured for rendering the results and reports of analysis carried out based on visual inputs, medical history information and measured parameters of the user, and wherein the visualization is provided by the system on a handheld computing device, and wherein the visualization module comprises biomechanics replay module, a comparative module, a simulation module and a summarization module.

According to an embodiment herein, the adaptive assessment module comprises a risk multiplier module, an activity specific stress modeling module, and an injury risk measurement module.

According to an embodiment herein, the risk multiplier module is run on the hardware processor in the computing system and configured to receive an input information from a user's biometric measurements, information on previous injuries, medical conditions and medical diagnosis information to compute a risk multiplier matrix for a given user, and wherein the risk multiplier module is configured to analyse the captured pictures or images or video, visual inputs of a user's postures and shape of limbs to assess the user's flexibility and strength, and wherein the flexibility is assessed based on a maximum angle of motion of a part of the body, strength and a maximum weight a held by the user for a preset period of time.

According to an embodiment herein, the activity specific stress modeling module is run on the hardware processor in the computing system and configured to measure a stress experienced by a body part of the user per unit time while performing a particular activity, sport, action or motion, and wherein the measured stress is compared with preset values populated from a historical data and past study to estimate the stress for calculating a personalized grade sensitivity matrix for the user in a particular activity, sport, action or motion.

According to an embodiment herein, the injury risk measurement module is run on the hardware processor in the computing system and configured to measure a potential risk of an injury based on historical data on injuries in an exercise or sport activity.

According to an embodiment herein, the visualization module comprises a biomechanics replay module, a comparative module, a simulation module, and a summarization module.

According to an embodiment herein, the biomechanics replay module is run on the hardware processor in the handheld computing system and configured to provide a replay option for the user to watch a recorded video of an activity performed by the user, and wherein the biomechanics replay module is further configured to provide comments and suggestions for correcting the actions performed in incorrect manner or potentially injury inducing manner.

According to an embodiment herein, the comparative module is run on the hardware processor in the handheld computing system and configured to compare the user's performance with the user's previous or past performance, other performers and optimum level of performance.

According to an embodiment herein, the simulation module is run on the hardware processor in the handheld computing system and configured to simulate an activity to be performed by the user to predict the performance and potential risk of injury to the user during the activity or exercise or sport activity.

According to an embodiment herein, a summarization module is run on the hardware processor in the handheld computing system and configured to provide a summary of the activity performed by the user, and wherein the summarization module is further configured to provide a rating to the user based on the performance during the activity, and wherein the rating is determined by analyzing the user performance and comparing the user performance with preset optimum performance levels.

According to an embodiment herein, the analytics module is configured to compute a plurality of results based on the captured pictures, or photos or videos or images of the user and prior data of the user's medical history, past illness and specific health related factors, and wherein the analytics module is configured to provides the results to the visualization module for rendering to the user.

According to an embodiment herein, the visualization module is configured to provide a single ranking to indicate the quality of the activity performed by the user.

According to an embodiment herein, a computer implemented method comprising instructions stored on a non-transitory computer readable storage medium and run a computing device provided with a hardware processor and memory for collecting, storing and analyzing specific form and posture related visual data from an athlete for predicting injury prone body parts and/or actions through data and visual analysis and simulations, is provided. The method comprising steps of capturing photos or images or pictures and/or videos of a user and a body part of the user during an exercise or sport activity, with an image capturing device; collecting specific form and posture related data from the captured picture or images or video of the user with a computing system or; capturing the poses and motion of a user performing an activity or action with a sensor module, and wherein the sensor module comprises a plurality of sensors, and wherein the sensor module is configured to detect and measure a time and magnitude of pressure exerted on the user during the exercise or sport activity and a magnitude of pressure released by the user during a plurality of actions performed by the user; creating an adaptive model with an adaptive assessment module provided in the computing system, and wherein the adaptive assessment module is run on a hardware processor provided in the computing system and configured to create an adaptive model based on the collected specific form and posture related data from the captured picture or images or video of the user visual data and pre-medical conditions provided by the user, and wherein the adaptive assessment module comprises a risk multiplier module, an activity specific stress modeling module and an injury risk measurement module; analysing an output data from the adaptive assessment module and rendering results and reports of analysis for predicting injury prone body parts and/or actions a handheld computing device, and wherein the hand held computing device is connected to the computing system through a wired or wireless network; analysing the data from the sensor module and the adaptive assessment module with an analytics module provided in the handheld computing device, and wherein the analytics module is configured to compute a plurality of results that are rendered to the user; and rendering the results and reports of analysis carried out based on visual inputs, medical history information and measured parameters of the user a visualization module provided in the handheld computing device, and wherein the visualization is provided by the system on a handheld computing device, and wherein the visualization module comprises biomechanics replay module, a comparative module, a simulation module and a summarization module.

According to an embodiment herein, the method further comprises collecting information on the pre-existing medical conditions of the user, biometric data that indicate the user's health and physical characteristics with a user input module; identifying posture, symmetry and body structure details of the user through visual recognition of bones, muscles or preset points on the user's body with the sensor module; quantitatively identifying load-bearing and stress-bearing ability of a body part of the user with the analytics module; calculating a personalized grade sensitivity matrix for the user and associated activity with a summarization module; and rendering and visualizing of data on a plurality of computing and display devices with the visualization module.

According to an embodiment herein, the step of quantitative identification of load-bearing and stress-bearing abilities of a body part comprises: calculating a risk multiplier matrix for the user from the user's biometric measurements, information on previous injuries, medical conditions and medical diagnosis information, and wherein photos and visual inputs of a user's postures and shape of limbs are used to assess the user's flexibility and strength, and wherein the flexibility is assessed based on the maximum angle of motion of a part of the body and wherein a strength is determined based on the maximum weight to be held by the user for a particular period of time; creating an activity specific stress modeling, wherein the activity specific stress modeling is created to measure the stress experienced by a body part of the user per unit time while performing a particular activity, sport, action or motion, and wherein the measured stress is compared with preset values populated by past studies and inferences are made for a particular user involved in a preset activity, sport, action or motion; and measuring the potential risk of an injury through population studies.

According to an embodiment herein, the step of rendering and visualizing of data on a plurality of computing and display devices comprises providing a biomechanics replay option for the user to watch a recorded video of an activity performed by the user, and wherein the biomechanics replay is done to provide comments and suggestions for correcting the actions performed in incorrect or potentially injury inducing manner; comparing the user's performance with the user's previous or past or historical performance, other performers and optimum level of performance; digitally simulating an activity to be performed by the user and predicting the performance and potential injury of risks to the user faces while performing the activity; providing a summary of the activity performed by the user; and providing a rating to the user based on the performance during the activity, and wherein the rating is determined by analyzing the user performance and comparing user performance with preset optimum performance levels.

According to an embodiment herein, a system and method for remote collection of visual data of an athlete's performance for the purpose of review by experts are provided. The embodiment also provides a method for decreasing the complexity of data analysis by reducing the number of variables used in the analyses. The visual data is also used in analyzing the characteristic parameters of athlete activities such as cumulative stress distribution of athlete's legs during an activity, fatigue-point measurement, to evaluate whether a usage of particular muscles are optimal etc. A plurality of sensors are attached at preset locations on the athlete's body to enable/perform a stress measurement, to provide an overview of the quality/efficiency of an activity.

According to one embodiment of the present disclosure, a system is provided for collecting specific form and posture related details based on image data or visual data from an athlete for analysis and evaluation of a stress or load bearing capability and performance of an athlete during an exercise/sport activity. The system comprises a user input module, a computing system and a hand held computing device. The computing system comprises an adaptive assessment module, a hardware processor. The handheld computing device is provided with an analytics module, visualization module, hardware processor, memory and an output module. The Adaptive Assessment Module is connected to the User and Visualization module and configured to assess the activities of the User and present results and reports to the Visualization module after processing the data. The Adaptive Assessment Module is also connected to Analytics module and configured to analyze the measured data.

According to an embodiment herein, an adaptive assessment module is provided. The adaptive assessment module is run on a hardware processor and configured to perform and create an adaptive modeling based on the visual data and pre-medical conditions provided by a user. The adaptive assessment module comprises a risk multiplier module, an activity specific stress modeling module and an injury risk measurement module. The risk multiplier module is run on the hardware processor and configured to receive or collect an input information from a user's biometric measurements, information on previous injuries, medical conditions and medical diagnosis information to compute a risk multiplier matrix for a particular user. The risk multiplier module is also configured to analyze the captured photos and visual inputs of a user's postures and shape of limbs to assess the user's flexibility and strength. The flexibility is assessed based on the maximum angle of motion of a part of the body. The strength is determined based on the maximum weight/stress a user is able to hold for a particular period of time (preset time period).

According to an embodiment herein, an activity specific stress modeling module is provided/included in the adaptive modeling module. The activity specific stress modeling module is run on a hardware processor and configured to measures the stress experienced by a body part of the user per unit time while performing a particular activity, sport, action or motion. The measured stress is compared with preset values populated based on past studies/historical data and inferences/predictions are made for a particular (given) user involved in a particular (specific) activity, sport, action or motion.

According to an embodiment herein, an injury risk measurement module is included/provided in the adaptive modeling module. The injury risk measurement module is run on a hardware processor and configured to measure the potential risk of an injury through population studies.

According to an embodiment herein, a visualization module is provided. The visualization module is run on the hardware and configured to render the results and reports of analysis carried out by the system based on visual inputs, medical history information and measured parameters of the user. The visualization output is provided by the system on a handheld computing device, which the user connects to the system through wired or wireless means. The visualization module comprises a biomechanics replay module, a comparative module, a simulation module and a summarization module. The biomechanics replay module is run on the hardware processor in the mobile computing device and configured to provide a replay option for the user to watch a recorded video of an activity performed by the user, along with comments and suggestions for improving the actions performed in an incorrect or potentially injury inducing manner. The comparative module is configured to compare a user's current performance (in real time) with the user's previous performance, other performers, optimum level of performance etc., stored in the memory. The simulation module is configured to simulate an activity to be performed by the user and predict the performance and potential injury risks faced by the user faces in performing the activity. The summarization module is configured to provide a summary report of the activity performed by the user and provide a rating to the user based on the performance during the activity. The rating is determined by analyzing the user performance and comparing the user performance with preset optimum performance levels.

According to an embodiment herein, a method is provided for collecting specific form and posture related data from a person's pictures taken at specific angles. The captured pictures/images of the athlete are segregated to a plurality of categories depending on the pose and the posture of the athlete in a picture. The captured pictures/images are used to provide the form and posture related analyses. The posture related analyses includes determining the arch and height of a person based on a picture of feet of a person, a strength of a person's back by identifying how much a person bends backwards based on visual information and parameters such as height and weight of a person to predict form and posture, determine the flexibility and potential stress points based on a video of the user while performing a preset activity etc.

According to an embodiment herein, a method is provided for extracting a form-related diagnosis from an athlete's video recording and establishing correlation between predicted analysis and actual observation. The embodiment also provides a system and method for predicting potential injury points in an athlete by analyzing the captured visual information and health parameters of the athlete.

According to an embodiment herein, the method comprises the following steps. The picture or video of user while performing an exercise/a sport activity is captured. The posture, symmetry and body structure details are identified through visual recognition of bones, muscles or preset points. A quantitative identification/estimation of load-bearing and stress-bearing capability of a body part is carried out. A personalized grade sensitivity matrix is calculated/computed to provide an intuitive representation of data and multiple outputs with different renderings of data.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a functional block diagram of a system for collecting specific form and posture related details based on image data or visual data from an athlete for analysis and evaluation of a stress or load bearing capability and performance of an athlete during an exercise/sport activity, according to an embodiment of the present invention.

FIG. 2 illustrates a functional block diagram of the adaptive assessment module in a computing system provided in a system for collecting specific form and posture related details based on image data or visual data from an athlete for analysis and evaluation of a stress or load bearing capability and performance of an athlete during an exercise/sport activity, according to an embodiment of the present invention.

FIG. 3 illustrates a functional block diagram of the visualization module, in a hand held computing device provided in a system for collecting specific form and posture related details based on image data or visual data from an athlete for analysis and evaluation of a stress or load bearing capability and performance of an athlete during an exercise/sport activity, according to an embodiment of the present invention.

FIG. 4 illustrates a flow chart explaining a method for collecting specific form and posture related details based on image data or visual data from an athlete for analysis and evaluation of a stress or load bearing capability and performance of an athlete during an exercise/sport activity, according to an embodiment of the present invention.

FIG. 5 illustrates a schematic representation of a plurality of actions performed by a user during an exercise/sport activity, in a computing system provided in a method for collecting specific form and posture related details based on image data or visual data from an athlete for analysis and evaluation of a stress or load bearing capability and performance of an athlete during an exercise/sport activity, according to an embodiment of the present invention.

Although the specific features of the embodiments herein are shown in some drawings and not in others. This is done for convenience only as each feature may be combined with any or all of the other features in accordance with the embodiment herein.

### DETAILED DESCRIPTION OF THE EMBODIMENTS HEREIN

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a system and method for collecting specific form and posture related visual data from an athlete for analysis. The present embodiments are also related to a system and method for extracting form related diagnosis from an athlete's video recording and establishing correlation between predicted analysis and actual observation.

According to an embodiment herein, a system is provided for collecting, storing and analyzing specific form and posture related visual data from an athlete for predicting injury prone body parts and/or actions through data and visual analysis and simulations. The system comprises an input module configured for capturing pictures or image or video of a user involved in an exercise or sport activity while performing the exercise and sport activity, and wherein the input module is an image capturing device, and wherein the image capturing device is a digital camera or a video camera. A computing system is configured for collecting specific form and posture related data from the captured picture or images or video of the user. A sensor module is provided in the computing system and configured for visually capturing the poses and motion of a user performing an activity or action, and wherein the sensor module comprises a plurality of sensors, and wherein the sensor module is configured to detect and measure a time and magnitude of pressure exerted on the user during the exercise or sport activity and a magnitude of pressure released by the user during a plurality of actions performed by the user. An adaptive assessment module is provided in the computing system to create an adaptive model, and wherein the adaptive assessment module is run on a hardware processor provided in the computing system and configured to create an adaptive model based on the collected specific form and posture related data from the captured picture or images or video of the user visual data and pre-medical conditions provided by the user, and wherein the adaptive assessment module comprises a risk multiplier module, an activity specific stress modeling module and an injury risk measurement module. A handheld computing device is configured for analysing an output data from the adaptive assessment module and rendering results and reports of analysis for predicting injury prone body parts and/or actions, and wherein the hand held computing device is connected to the computing system through a wired or wireless network. An analytics module is provided in the handheld computing device and configured for analysing the data from the sensor module and the adaptive assessment module, and wherein the analytics module is configured to compute a plurality of results that are rendered to the user. A visualization module is provided in the handheld computing device and configured for rendering the results and reports of analysis carried out based on visual inputs, medical history information and measured parameters of the user, and wherein the visualization is provided by the system on a handheld computing device, and wherein the visualization module comprises biomechanics replay module, a comparative module, a simulation module and a summarization module.

According to an embodiment herein, the adaptive assessment module comprises a risk multiplier module, an activity specific stress modeling module, and an injury risk measurement module.

According to an embodiment herein, the risk multiplier module is run on the hardware processor in the computing system and configured to receive an input information from a user's biometric measurements, information on previous injuries, medical conditions and medical diagnosis information to compute a risk multiplier matrix for a given user, and wherein the risk multiplier module is configured to analyse the captured pictures or images or video, visual inputs of a user's postures and shape of limbs to assess the user's flexibility and strength, and wherein the flexibility is assessed based on a maximum angle of motion of a part of the body, strength and a maximum weight a held by the user for a preset period of time.

According to an embodiment herein, the activity specific stress modeling module is run on the hardware processor in the computing system and configured to measure a stress experienced by a body part of the user per unit time while performing a particular activity, sport, action or motion, and wherein the measured stress is compared with preset values populated from a historical data and past study to estimate the stress for calculating a personalized grade sensitivity matrix for the user in a particular activity, sport, action or motion.

According to an embodiment herein, the injury risk measurement module is run on the hardware processor in the computing system and configured to measure a potential risk of an injury based on historical data on injuries in an exercise or sport activity.

According to an embodiment herein, the visualization module comprises a biomechanics replay module, a comparative module, a simulation module, and a summarization module.

According to an embodiment herein, the biomechanics replay module is run on the hardware processor in the handheld computing system and configured to provide a replay option for the user to watch a recorded video of an activity performed by the user, and wherein the biomechanics replay module is further configured to provide comments and suggestions for correcting the actions performed in incorrect manner or potentially injury inducing manner.

According to an embodiment herein, the comparative module is run on the hardware processor in the handheld computing system and configured to compare the user's performance with the user's previous or past performance, other performers and optimum level of performance.

According to an embodiment herein, the simulation module is run on the hardware processor in the handheld computing system and configured to simulate an activity to be performed by the user to predict the performance and potential risk of injury to the user during the activity or exercise or sport activity.

According to an embodiment herein, a summarization module is run on the hardware processor in the handheld computing system and configured to provide a summary of the activity performed by the user, and wherein the summarization module is further configured to provide a rating to the user based, on the performance during the activity, and wherein the rating is determined by analyzing the user performance and comparing the user performance with preset optimum performance levels.

According to an embodiment herein, the analytics module is configured to compute a plurality of results based on the captured pictures, or photos or videos or images of the user and prior data of the user's medical history, past illness and specific health related factors, and wherein the analytics module is configured to provides the results to the visualization module for rendering to the user.

According to an embodiment herein, the visualization module is configured to provide a single ranking to indicate the quality of the activity performed by the user.

According to an embodiment herein, a computer implemented method comprising instructions stored on a non-transitory computer readable storage medium and run a computing device provided with a hardware processor and memory for collecting, storing and analyzing specific form and posture related visual data from an athlete for predicting injury prone body parts and/or actions through data and visual analysis and simulations, is provided. The method comprising steps of capturing photos or images or pictures and/or videos of a user and a body part of the user during an exercise or sport activity, with an image capturing device; collecting specific form and posture related data from the captured picture or images or video of the user with a computing system or; capturing the poses and motion of a user performing an activity or action with a sensor module, and wherein the sensor module comprises a plurality of sensors, and wherein the sensor module is configured to detect and measure a time and magnitude of pressure exerted on the user during the exercise or sport activity and a magnitude of pressure released by the user during a plurality of actions performed by the user; creating an adaptive model with an adaptive assessment module provided in the computing system, and wherein the adaptive assessment module is run on a hardware processor provided in the computing system and configured to create an adaptive model based on the collected specific form and posture related data from the captured picture or images or video of the user visual data and pre-medical conditions provided by the user, and wherein the adaptive assessment module comprises a risk multiplier module, an activity specific stress modeling module and an injury risk measurement module; analysing an output data from the adaptive assessment module and rendering results and reports of analysis for predicting injury prone body parts and/or actions a handheld computing device, and wherein the hand held computing device is connected to the computing system through a wired or wireless network; analysing the data from the sensor module and the adaptive assessment module with an analytics module provided in the handheld computing device, and wherein the analytics module is configured to compute a plurality of results that are rendered to the user; and rendering the results and reports of analysis carried out based on visual inputs, medical history information and measured parameters of the user a visualization module provided in the handheld computing device, and wherein the visualization is provided by the system on a handheld computing device, and wherein the visualization module comprises biomechanics replay module, a comparative module, a simulation module and a summarization module.

According to an embodiment herein, the method further comprises collecting information on the pre-existing medical conditions of the user, biometric data that indicate the user's health and physical characteristics with a user input module; identifying posture, symmetry and body structure details of the user through visual recognition of bones, muscles or preset points on the user's body with the sensor module; quantitatively identifying load-bearing and stress-bearing ability of a body part of the user with the analytics module; calculating a personalized grade sensitivity matrix for the user and associated activity with a summarization module; and rendering and visualizing of data on a plurality of computing and display devices with the visualization module.

According to an embodiment herein, the step of quantitative identification of load-bearing and stress-bearing abilities of a body part comprises: calculating a risk multiplier matrix for the user from the user's biometric measurements, information on previous injuries, medical conditions and medical diagnosis information, and wherein photos and visual inputs of a user's postures and shape of limbs are used to assess the user's flexibility and strength, and wherein the flexibility is assessed based on the maximum angle of motion of a part of the body and wherein a strength is determined based on the maximum weight to be held by the user for a particular period of time; creating an activity specific stress modeling, wherein the activity specific stress modeling is created to measure the stress experienced by a body part of the user per unit time while performing a particular activity, sport, action or motion, and wherein the measured stress is compared with preset values populated by past studies and inferences are made for a particular user involved in a preset activity, sport, action or motion; and measuring the potential risk of an injury through population studies.

According to an embodiment herein, the step of rendering and visualizing of data on a plurality of computing and display devices comprises providing a biomechanics replay option for the user to watch a recorded video of an activity performed by the user, and wherein the biomechanics replay is done to provide comments and suggestions for correcting the actions performed in incorrect or potentially injury inducing manner; comparing the user's performance with the user's previous or past or historical performance, other performers and optimum level of performance; digitally simulating an activity to be performed by the user and predicting the performance and potential injury of risks to the user faces while performing the activity; providing a summary of the activity performed by the user; and providing a rating to the user based on the performance during the activity, and wherein the rating is determined by analyzing the user performance and comparing user performance with preset optimum performance levels.

According to an embodiment herein, a system and method for remote collection of visual data of an athlete's performance for the purpose of review by experts are provided. The embodiment also provides a method for decreasing the complexity of data analysis by reducing the number of variables used in the analyses. The visual data is also used in analyzing the characteristic parameters of athlete activities such as cumulative stress distribution of athlete's legs during an activity, fatigue-point measurement, to evaluate whether a usage of particular muscles are optimal etc. A plurality of sensors are attached at preset locations on the athlete's body to enable/perform a stress measurement, to provide an overview of the quality/efficiency of an activity.

According to one embodiment of the present disclosure, a system is provided for collecting specific form and posture related details based on image data or visual data from an athlete for analysis and evaluation of a stress or load bearing capability and performance of an athlete during an exercise/sport activity. The system comprises a user input module, a computing system and a hand held computing device. The computing system comprises an adaptive assessment module, a hardware processor. The handheld computing device is provided with an analytics module, visualization module, hardware processor, memory and an output module. The Adaptive Assessment Module is connected to the User and Visualization module and configured to assess the activities of the User and present results and reports to the Visualization module after processing the data. The Adaptive Assessment Module is also connected to Analytics module and configured to analyze the measured data.

According to an embodiment herein, an adaptive assessment module is provided. The adaptive assessment module is run on a hardware processor and configured to perform and create an adaptive modeling based on the visual data and pre-medical conditions provided by a user. The adaptive assessment module comprises a risk multiplier module, an activity specific stress modeling module and an injury risk measurement module. The risk multiplier module is run on the hardware processor and configured to receive or collect an input information from a user's biometric measurements, information on previous injuries, medical conditions and medical diagnosis information to compute a risk multiplier matrix for a particular user. The risk multiplier module is also configured to analyze the captured photos and visual inputs of a user's postures and shape of limbs to assess the user's flexibility and strength. The flexibility is assessed based on the maximum angle of motion of a part of the body. The strength is determined based on the maximum weight/stress a user is able to hold for a particular period of time (preset time period).

According to an embodiment herein, an activity specific stress modeling module is provided/included in the adaptive modeling module. The activity specific stress modeling module is run on a hardware processor and configured to measures the stress experienced by a body part of the user per unit time while performing a particular activity, sport, action or motion. The measured stress is compared with preset values populated based on past studies/historical data and inferences/predictions are made for a particular (given) user involved in a particular (specific) activity, sport, action or motion.

According to an embodiment herein, an injury risk measurement module is included/provided in the adaptive modeling module. The injury risk measurement module is run on a hardware processor and configured to measure the potential risk of an injury through population studies.

According to an embodiment herein, a visualization module is provided. The visualization module is run on the hardware and configured to render the results and reports of analysis carried out by the system based on visual inputs, medical history information and measured parameters of the user. The visualization output is provided by the system on a handheld computing device, which the user connects to the system through wired or wireless means. The visualization module comprises a biomechanics replay module, a comparative module, a simulation module and a summarization module. The biomechanics replay module is run on the hardware processor in the mobile computing device and configured to provide a replay option for the user to watch a recorded video of an activity performed by the user, along with comments and suggestions for improving the actions performed in an incorrect or potentially injury inducing manner. The comparative module is configured to compare a user's current performance (in real time) with the user's previous performance, other performers, optimum level of performance etc., stored in the memory. The simulation module is configured to simulate an activity to be performed by the user and predict the performance and potential injury risks faced by the user faces in performing the activity. The summarization module is configured to provide a summary report of the activity performed by the user and provide a rating to the user based on the performance during the activity. The rating is determined by analyzing the user performance and comparing the user performance with preset optimum performance levels.

According to an embodiment herein, a method is provided for collecting specific form and posture related data from a person's pictures taken at specific angles. The captured pictures/images of the athlete are segregated to a plurality of categories depending on the pose and the posture of the athlete in a picture. The captured pictures/images are used to provide the form and posture related analyses. The posture related analyses includes determining the arch and height of a person based on a picture of feet of a person, a strength of a person's back by identifying how much a person bends backwards based on visual information and parameters such as height and weight of a person to predict form and posture, determine the flexibility and potential stress points based on a video of the user while performing a preset activity etc.

According to an embodiment herein, a method is provided for extracting a form-related diagnosis from an athlete's video recording and establishing correlation between predicted analysis and actual observation. The embodiment also provides a system and method for predicting potential injury points in an athlete by analyzing the captured visual information and health parameters of the athlete.

According to an embodiment herein, the method comprises the following steps. The picture or video of user while performing an exercise/a sport activity is captured. The posture, symmetry and body structure details are identified through visual recognition of bones, muscles or preset points. A quantitative identification/estimation of load-bearing and stress-bearing capability of a body part is carried out. A personalized grade sensitivity matrix is calculated/computed to provide an intuitive representation of data and multiple outputs with different renderings of data.

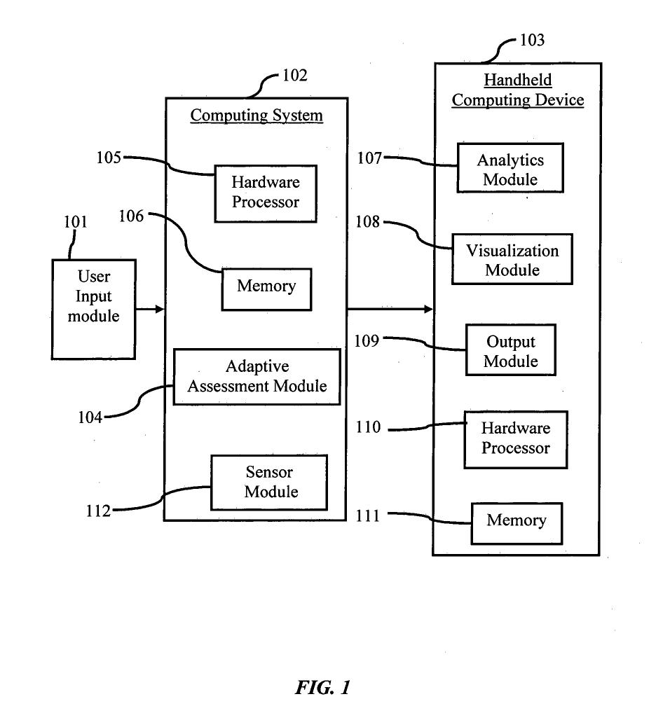

FIG. 1 illustrates a functional block diagram of a system for collecting specific form and posture related details based on image data or visual data from an athlete for analysis and evaluation of a stress or load bearing capability and performance of an athlete during an exercise/sport activity, according to an embodiment of the present invention.

With respect to FIG. 1, the system comprises a user input module 101, a computing system 102 and a hand held computing device 103. The computing system 102 comprises an adaptive assessment module 104, a hardware processor 105 sensor module 112, and a memory 106. The handheld computing device 103 is provided with an analytics module 107, visualization module 108, an output module 109, hardware processor 110 and memory 111. The adaptive assessment module 104 is connected to the user input module 101 and visualization module 108 and configured to assess the activities of the user and present results and reports to the visualization module 108 after processing the data. The adaptive assessment module 104 is also connected to analytics module and configured to analyze the measured data.

According to an embodiment herein, an adaptive assessment module 104 is provided. The adaptive assessment module is run on a hardware processor 105 and configured to perform and create an adaptive modeling based on the visual data and pre-medical conditions provided by a user. The adaptive assessment module comprises a risk multiplier module, an activity specific stress modeling module and an injury risk measurement module. The risk multiplier module is run on the hardware processor and configured to receive or collect an input information from a user's biometric measurements, information on previous injuries, medical conditions and medical diagnosis information to compute a risk multiplier matrix for a particular user. The risk multiplier module is also configured to analyze the captured photos and visual inputs of a user's postures and shape of limbs to assess the user's flexibility and strength. The flexibility is assessed based on the maximum angle of motion of a part of the body. The strength is determined based on the maximum weight/stress a user is able to hold for a particular period of time (preset time period).

According to an embodiment herein, an activity specific stress modeling module is provided/included in the adaptive modeling module. The activity specific stress modeling module is run on a hardware processor and configured to measures the stress experienced by a body part of the user per unit time while performing a particular activity, sport, action or motion. The measured stress is compared with preset values populated based on past studies/historical data and inferences/predictions are made for a particular (given) user involved in a particular (specific) activity, sport, action or motion.

According to an embodiment herein, an injury risk measurement module is included/provided in the adaptive modeling module. The injury risk measurement module is run on a hardware processor and configured to measure the potential risk of an injury through population studies.

According to an embodiment herein, a visualization module is provided. The visualization module is run on the hardware and configured to render the results and reports of analysis carried out by the system based on visual inputs, medical history information and measured parameters of the user. The visualization output is provided by the system on a handheld computing device, which the user connects to the system through wired or wireless means. The visualization module comprises a biomechanics replay module, a comparative module, a simulation module and a summarization module. The biomechanics replay module is run on the hardware processor in the mobile computing device and configured to provide a replay option for the user to watch a recorded video of an activity performed by the user, along with comments and suggestions for improving the actions performed, in an incorrect or potentially injury inducing manner. The comparative module is configured to compare a user's current performance (in real time) with the user's previous performance, other performers, optimum level of performance etc., stored in the memory. The simulation module is configured to simulate an activity to be performed by the user and predict the performance and potential injury risks faced by the user faces in performing the activity. The summarization module is configured to provide a summary report of the activity performed by the user and provide a rating to the user based on the performance during the activity. The rating is determined by analyzing the user performance and comparing the user performance with preset optimum performance levels.

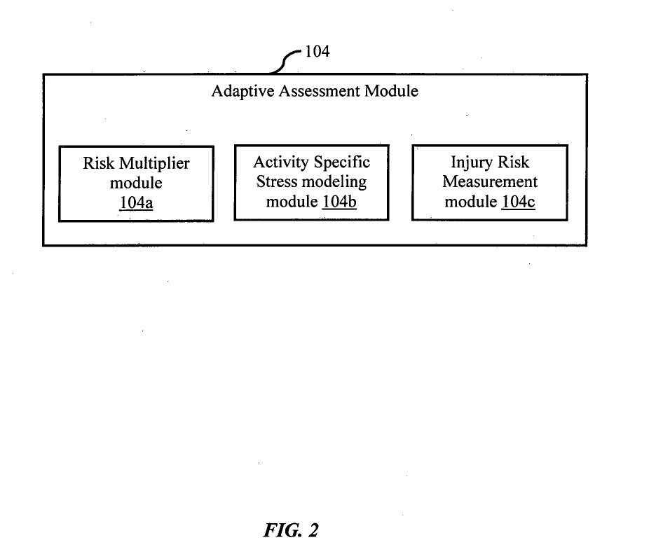

FIG. 2 illustrates a functional block diagram of the adaptive assessment module in a computing system provided in a system for collecting specific form and posture related details based on image data or visual data from an athlete for analysis and evaluation of a stress or load bearing capability and performance of an athlete during an exercise/sport activity, according to an embodiment of the present invention. With respect to FIG. 2, the adaptive assessment module 104 is run on a hardware processor 105 and configured to perform and create an adaptive modeling based on the visual data and pre-medical conditions provided by a user. The adaptive assessment module 104 comprises a risk multiplier module 104a, an activity specific stress modeling module 104b and an injury risk measurement module 104c. The risk multiplier module 104a is run on the hardware processor and configured to receive or collect an input information from a user's biometric measurements, information on previous injuries, medical conditions and medical diagnosis information to compute a risk multiplier matrix for a particular user. The risk multiplier module is also configured to analyze the captured photos and visual inputs of a user's postures and shape of limbs to assess the user's flexibility and strength. The flexibility is assessed based on the maximum angle of motion of a part of the body. The strength is determined based on the maximum weight/stress a user is able to hold for a particular period of time (preset time period).

According to an embodiment herein, an activity specific stress modeling module 104b is provided/included in the adaptive modeling module. The activity specific stress modeling module is run on a hardware processor and configured to measures the stress experienced by a body part of the user per unit time while performing a particular activity, sport, action or motion. The measured stress is compared with preset values populated based on past studies/historical data and inferences/predictions are made for a particular (given) user involved in a particular (specific) activity, sport, action or motion.

According to an embodiment herein, an injury risk measurement module 104c is included/provided in the adaptive modeling module. The injury risk measurement module is run on a hardware processor and configured to measure the potential risk of an injury through population studies.

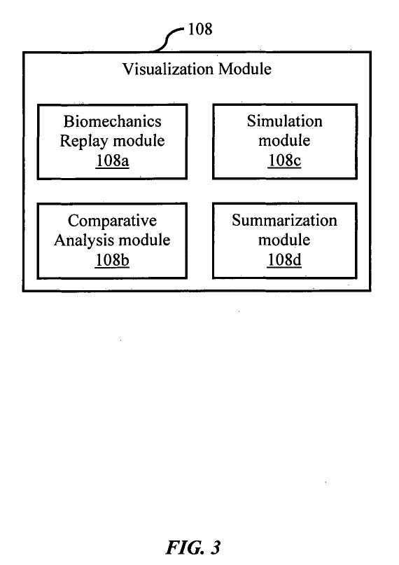

FIG. 3 illustrates a functional block diagram of the visualization module 108, in a hand held computing device 103 provided in a system for collecting specific form and posture related details based on image data or visual data from an athlete for analysis and evaluation of a stress or load bearing capability and performance of an athlete during an exercise/sport activity, according to an embodiment of the present invention. With respect to the visualization module is run on the hardware and configured to render the results and reports of analysis carried out by the system based on visual inputs, medical history information and measured parameters of the user. The visualization output is provided by the system on a handheld computing device, which the user connects to the system through wired or wireless means. The visualization module 108 comprises a biomechanics replay module 108a, a comparative module 108b, a simulation module 108c and a summarization module 108d. The biomechanics replay module is run on the hardware processor in the mobile computing device and configured to provide a replay option for the user to watch a recorded video of an activity performed by the user, along with comments and suggestions for improving the actions performed in an incorrect or potentially injury inducing manner. The comparative module is configured to compare a user's current performance (in real time) with the user's previous performance, other performers, optimum level of performance etc., stored in the memory. The simulation module is configured to simulate an activity to be performed by the user and predict the performance and potential injury risks faced by the user faces in performing the activity. The summarization module is configured to provide a summary report of the activity performed by the user and provide a rating to the user based on the performance during the activity. The rating is determined by analyzing the user performance and comparing the user performance with preset optimum performance levels.

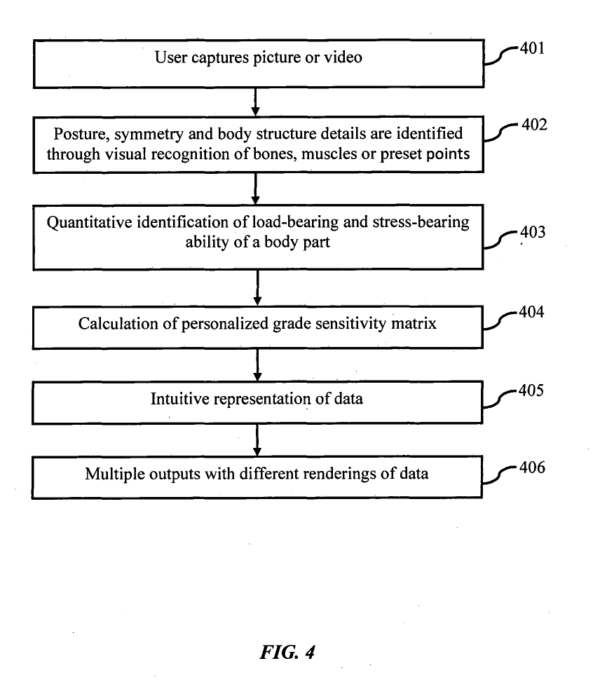

FIG. 4 illustrates a flow chart explaining a method for collecting specific form and posture related details based on image data or visual data from an athlete for analysis and evaluation of a stress or load bearing capability and performance of an athlete during an exercise/sport activity, according to an embodiment of the present invention. With respect to FIG. 4, the picture or video of user while performing an exercise/a sport activity is captured (401). The posture, symmetry and body structure details are identified through visual recognition of bones, muscles or preset points (402). A quantitative identification/estimation of load-bearing and stress-bearing capability of a body part is carried out (403). A personalized grade sensitivity matrix is calculated/computed (404). An intuitive representation of data is provided based on calculated/computed personalized grade sensitivity matrix (405). The plurality of data are rendered in and the plurality of data are mutually different data.

According to an embodiment herein, a method is provided for collecting specific form and posture related data from a person's pictures taken at specific angles. The captured pictures/images of the athlete are segregated to a plurality of categories depending on the pose and the posture of the athlete in a picture. The captured pictures/images are used to provide the form and posture related analyses. The posture related analyses includes determining the arch and height of a person based on a picture of feet of a person, a strength of a person's back by identifying how much a person bends backwards based on visual information and parameters such as height and weight of a person to predict form and posture, determine the flexibility and potential stress points based on a video of the user while performing a preset activity etc.

According to an embodiment herein, a method is provided for extracting a form-related diagnosis from an athlete's video recording and establishing correlation between predicted analysis and actual observation. The embodiment also provides a system and method for predicting potential injury points in an athlete by analyzing the captured visual information and health parameters of the athlete.

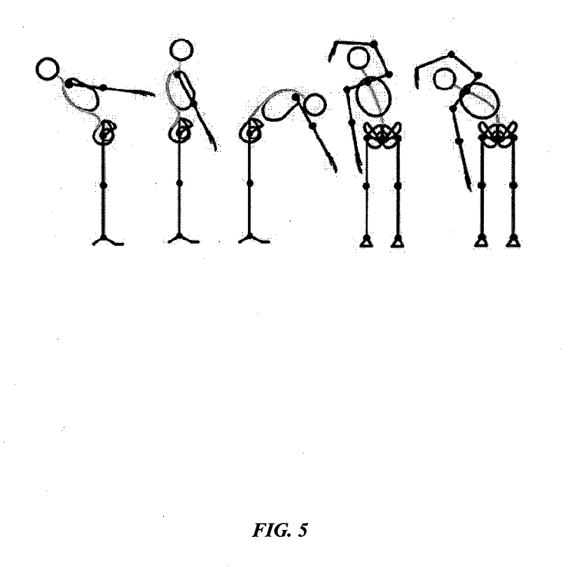

FIG. 5 illustrates a schematic representation of a plurality of actions performed by a user during an exercise/sport activity, in a computing system provided in a method for collecting specific form and posture related details based on image data or visual data from an athlete for analysis and evaluation of a stress or load bearing capability and performance of an athlete during an exercise/sport activity, according to an embodiment of the present invention.

The advantages of the embodiments herein comprise extracting form-related diagnosis from an athlete's video recording, pictures and establishing correlation between predicted analysis and actual observation. The embodiments herein also provide a system and method for predicting an athlete's potential injury points through visual inputs of athletes' body parts. The embodiments provide athletes with an option to prevent a possible injury without actually performing the activity or physically simulating the activity.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the disclosure with modifications. However, all such modifications are deemed to be within the scope of the appended claims.

It is also to be understood that the following claims are intended to cover all of the generic and specific features of the embodiments described herein and all the statements of the scope of the embodiments which as a matter of language might be said to fall there between.

What is claimed is:

1. A system for collecting, storing and analyzing specific form and posture related visual data from an athlete for predicting injury prone body parts and/or actions through data and visual analysis and simulations, the system comprising:
an input module configured for capturing pictures or image or video of a user involved in an exercise or sport activity while performing the exercise and sport activity, and wherein the input module is an image capturing device, and wherein the image capturing device is a digital camera or a video camera;
a computing system configured for collecting specific form and posture related data from the captured picture or images or video of the user;
a sensor module provided in the computing system and configured for visually capturing the poses and motion of a user performing an activity or action, and wherein the sensor module comprises a plurality of sensors, and wherein the sensor module is configured to detect and measure a time and magnitude of pressure exerted on the user during the exercise or sport activity and a magnitude of pressure released by the user during a plurality of actions performed by the user;
an adaptive assessment module provided in the computing system to create an adaptive model, and wherein the adaptive assessment module is run on a hardware processor provided in the computing system and configured to create an adaptive model based on the collected specific form and posture related data from the captured picture or images or video of the user visual data and pre-medical conditions provided by the user, and wherein the adaptive assessment module comprises a risk multiplier module, an activity specific stress modeling module and an injury risk measurement module;
a handheld computing device configured for analysing an output data from the adaptive assessment module and rendering results and reports of analysis for predicting injury prone body parts and/or actions, and wherein the hand held computing device is connected to the computing system through a wired or wireless network;
an analytics module provided in the handheld computing device and configured for analysing the data from the sensor module and the adaptive assessment module, and wherein the analytics module is configured to compute a plurality of results that are rendered to the user; and
a visualization module provided in the handheld computing device and configured for rendering the results and reports of analysis carried out based on visual inputs, medical history information and measured parameters of the user, and wherein the visualization is provided by the system on a handheld computing device, and wherein the visualization module comprises biomechanics replay module, a comparative module, a simulation module and a summarization module.

2. The device according to claim 1, wherein the adaptive assessment module comprises:
a risk multiplier module run on the hardware processor in the computing system and configured to receive an input information from a user's biometric measurements, information on previous injuries, medical conditions and medical diagnosis information to compute a risk multiplier matrix for a given user, and wherein the risk multiplier module is configured to analyse the captured pictures or images or video, visual inputs of a user's postures and shape of limbs to assess the user's flexibility and strength, and wherein the flexibility is assessed based on a maximum angle of motion of a part of the body, strength and a maximum weight a held by the user for a preset period of time;
an activity specific stress modeling module run on the hardware processor in the computing system and configured to measure a stress experienced by a body part of the user per unit time while performing a particular activity, sport, action or motion, and wherein the measured stress is compared with preset values populated from a historical data and past study to estimate the stress for calculating a personalized grade sensitivity matrix for the user in a particular activity, sport, action or motion; and
an injury risk measurement module run on the hardware processor in the computing system and configured to measure a potential risk of an injury based on historical data on injuries in an exercise or sport activity.

3. The device according to claim 1, wherein the visualization module comprises:
a biomechanics replay module run on the hardware processor in the handheld computing system and configured to provide a replay option for the user to watch a recorded video of an activity performed by the user, and wherein the biomechanics replay module is further configured to provide comments and suggestions for correcting the actions performed in incorrect manner or potentially injury inducing manner;
a comparative module run on the hardware processor in the handheld computing system and configured to compare the user's performance with the user's previous or past performance, other performers and optimum level of performance;
a simulation module run on the hardware processor in the handheld computing system and configured to simulate an activity to be performed by the user to predict the performance and potential risk of injury to the user during the activity or exercise or sport activity; and,
a summarization module run on the hardware processor in the handheld computing system and configured to provide a summary of the activity performed by the user, and wherein the summarization module is further configured to provide a rating to the user based on the performance during the activity, and wherein the rating is determined by analyzing the user performance and comparing the user performance with preset optimum performance levels.

4. The device according to claim 1, wherein the analytics module is configured to compute a plurality of results based on the captured pictures, or photos or videos or images of the user and prior data of the user's medical history, past illness and specific health related factors, and wherein the analytics module is configured to provides the results to the visualization module for rendering to the user.

5. The device according to claim 1, wherein the visualization module is configured to provides a single ranking to indicate the quality of the activity performed by the user.

6. A computer implemented method comprising instructions stored on a non-transitory computer readable storage medium and run a computing device provided with a hardware processor and memory for collecting, storing and analyzing specific form and posture related visual data from an athlete for predicting injury prone body parts and/or actions through data and visual analysis and simulations, the method comprising steps of:
capturing photos or images or pictures and/or videos of a user and a body part of the user during an exercise or sport activity, with an image capturing device;
collecting specific form and posture related data from the captured picture or images or video of the user with a computing system or;
capturing the poses and motion of a user performing an activity or action with a sensor module, and wherein the sensor module comprises a plurality of sensors, and wherein the sensor module is configured to detect and measure a time and magnitude of pressure exerted on the user during the exercise or sport activity and a magnitude of pressure released by the user during a plurality of actions performed by the user;
creating an adaptive model with an adaptive assessment module provided in the computing system, and wherein the adaptive assessment module is run on a hardware processor provided in the computing system and configured to create an adaptive model based on the collected specific form and posture related data from the captured picture or images or video of the user visual data and pre-medical conditions provided by the user, and wherein the adaptive assessment module comprises a risk multiplier module, an activity specific stress modeling module and an injury risk measurement module;
analysing an output data from the adaptive assessment module and rendering results and reports of analysis for predicting injury prone body parts and/or actions a handheld computing device, and wherein the hand held computing device is connected to the computing system through a wired or wireless network;
analysing the data from the sensor module and the adaptive assessment module with an analytics module provided in the handheld computing device, and wherein the analytics module is configured to compute a plurality of results that are rendered to the user; and
rendering the results and reports of analysis carried out based on visual inputs, medical history information and measured parameters of the user a visualization module provided in the handheld computing device, and wherein the visualization is provided by the system on a handheld computing device, and wherein the visualization module comprises biomechanics replay module, a comparative module, a simulation module and a summarization module.

7. The method according to claim 6, further comprises:
collecting information on the pre-existing medical conditions of the user, biometric data that indicate the user's health and physical characteristics with a user input module;
identifying posture, symmetry and body structure details of the user through visual recognition of bones, muscles or preset points on the user's body with the sensor module;
quantitatively identifying load-bearing and stress-bearing ability of a body part of the user with the analytics module;
calculating a personalized grade sensitivity matrix for the user and associated activity with a summarization module; and,
rendering and visualizing of data on a plurality of computing and display devices with the visualization module.

8. The method according to claim 6, wherein the step of quantitative identification of load-bearing and stress-bearing abilities of a body part comprises:
calculating a risk multiplier matrix for the user from the user's biometric measurements, information on previous injuries, medical conditions and medical diagnosis information, and wherein photos and visual inputs of a user's postures and shape of limbs are used to assess the user's flexibility and strength, and wherein the flexibility is assessed based on the maximum angle of motion of a part of the body and wherein a strength is determined based on the maximum weight to be held by the user for a particular period of time;
creating an activity specific stress modeling, wherein the activity specific stress modeling is created to measure the stress experienced by a body part of the user per unit time while performing a particular activity, sport, action or motion, and wherein the measured stress is compared with preset values populated by past studies and inferences are made for a particular user involved in a preset activity, sport, action or motion; and,
measuring the potential risk of an injury through population studies.

9. The method according to claim 6, wherein the step of rendering and visualizing of data on a plurality of computing and display devices comprises:
providing a biomechanics replay option for the user to watch a recorded video of an activity performed by the user, and wherein the biomechanics replay is done to provide comments and suggestions for correcting the actions performed in incorrect or potentially injury inducing manner;
comparing the user's performance with the user's previous or past or historical performance, other performers and optimum level of performance;
digitally simulating an activity to be performed by the user and predicting the performance and potential injury of risks to the user faces while performing the activity;
providing a summary of the activity performed by the user; and
providing a rating to the user based on the performance during the activity, and wherein the rating is determined by analyzing the user performance and comparing user performance with preset optimum performance levels.
